(19) United States
(12) Patent Application Publication
(10) Pub. No.: US 2022/0273205 A1
(43) Pub. Date: Sep. 1, 2022

(54) DEVICE AND METHOD FOR MEASURING BLOOD AND SKIN COMPONENTS

(71) Applicant: Shani Biotechnologies LLC, Austin, TX (US)

(72) Inventor: Sanjay Gokhale, Arlington, TX (US)

(73) Assignee: Shani Biotechnologies LLC, Austin, TX (US)

(21) Appl. No.: 17/749,269

(22) Filed: May 20, 2022

Related U.S. Application Data
(63) Continuation-in-part of application No. 17/542,765, filed on Dec. 6, 2021.
Application No. 17/542,765 is a division of application No. 17/317,171, filed on May 11, 2021, now Pat. No. 11,191,460.
(60) Provisional application No. 63/072,504, filed on Aug. 31, 2020.
(60) Provisional application No. 63/052,115, filed on Jul. 15, 2020.

(51) Int. Cl. A61B 5/1455; A61B 5/00; A61B 5/103

(52) U.S. Cl. CPC A61B 5/14551; A61B 5/443; A61B 5/1032; A61B 5/748; A61B 5/7435

(57) ABSTRACT

The present disclosure is directed to a method and device to determine blood and/or skin constituents of a mammal. The device includes a plurality of light emitting diodes, one or more sensors and a processor.

### CROSS REFERENCE TO RELATED APPLICATION

This application is a continuation-in-part of co-pending application Ser. No. 17/542,765, filed Dec. 6, 2021, which is a divisional application of co-pending application Ser. No. 17/317,171, filed on May 11, 2021, now U.S. Pat. No. 11,191,460, which claims the benefit of both of U.S. Provisional Patent Application Ser. No. 63/052,115 filed on Jul. 15, 2020 and U.S. Provisional Patent Application Ser. No. 63/072,504 filed on Aug. 31, 2020, the entire contents of each of which are herein incorporated by reference.

### BACKGROUND OF THE DISCLOSURE

The measurement and monitoring of blood constituents such as hemoglobin (Hb) and its species such as oxy-hemoglobin (O-Hb), bilirubin, and other skin constituents such as melanin, is often an invasive procedure. The blood draw can cause discomfort to the patient and increases the risk of infection. The adverse effects of constant and often difficult blood draws on newborns and infants are even more pronounced. Frequent and constant blood and/or skin constituent measurements are clinically indicated in a wide variety of conditions such as (but not limited to) anemia, emergency and critical care including patients infected with Coronavirus Disease of 2019 (COVID-19), hemoglobinopathies such as sickle cell disease (hemoglobin-S), hematological malignancies, peri-operative management after major surgeries such as spine surgery, jaundice, liver diseases, alcoholics, and critical care patients with multiorgan failures and hemoglobinopathies such as sickle cell disease (hemoglobin-S), and those who need frequent blood transfusions, etc.

In addition, these parameters are part of routine health maintenance in healthy subjects and are increasingly being used to monitor athletic performance.

Thus, frequent measurements of blood and/or skin constituents have wide applications in healthy adults and children, athletes, patients recovering from various illnesses, as well as patients admitted in the hospital setting. Although the available invasive techniques are established, they require the presence of medical personnel and laboratory equipment. These requirements drive up the cost, as well as the amount of time for the results to be available for the clinical use.

The measurement and monitoring of blood constituents such as bilirubin [bilirubin mg/dL or mg/100 mL] is typically an invasive procedure with a blood draw, which can cause discomfort to the patient and increases the risk of infection. The adverse effects of constant and often difficult blood draws on newborns and infants are even more pronounced.

High bilirubin or jaundice is associated with liver disease and is a marker for severity of liver dysfunction. Elevated levels of bilirubin in blood are seen in liver damage due to many conditions like alcoholic liver disease, nonalcoholic steatorrhic hepatitis [NASH], infections affecting liver such as Hepatitis B or C, and drug induced liver damage etc. High bilirubin is also seen in conditions like sickle cell anemia and thalassemia or other hemolytic anemias.

The bilirubin can be transferred from plasma to skin and there is leakage of bilirubin-albumin complexes into extravascular spaces and precipitation of bilirubin acid in phospholipid membranes of skin cells can occur. Thus, measurement of the yellow color of the skin may be a better predictor of severity of jaundice in a patient, such as a newborn, as compared to serum bilirubin concentration.

High blood bilirubin causes brain damage in newborn babies and therefore close and frequent monitoring of blood bilirubin is necessary in babies suffering from jaundice.

Although the available invasive techniques are established, they require the presence of medical personnel and laboratory equipment. Typical laboratory methods include the Jendrassik-Grof-Diazo reagent method, and an enzymatic method which employs bilirubin oxidase. These requirements drive up the cost, as well as the amount of time for the results to be available for the clinical use.

There are non-invasive methods, however they suffer from variability and limitations, because of the overlapping of the hemoglobin spectral values and the effect melanin concentration can have on typical spectral methods. Bilirubin exists in two forms Unconjugated or Indirect and conjugated or direct bilirubin. However, both share the same absorption spectra.

Typical bilirubin meters work by directing light into the skin of the patient (e.g., neonate) and measuring the intensity of specific wavelength that is returned. The meter analyzes the spectrum of optical signal reflected from the neonate's subcutaneous tissues. These optical signals are converted to electrical signal by a photocell. These are analyzed by a microprocessor to generate a serum bilirubin value. However, accuracy of these meters is low, especially with patients having a high melanin concentration.

One embodiment of the present disclosure is directed to the measurement of bilirubin concentration using reflectance spectroscopy. The advantages of this technology are the use of a device, as described herein, to non-invasively measure bilirubin concentration in real time, and a method for continuous (or nearly continuous) non-invasive monitoring.

A skin constituent that can be measured is melanin.

In the visible range, the main chromophores of human skin are hemoglobin and melanin. Hemoglobin is found in the microvascular network of the dermis, typically up to about 0.5 mm below the skin surface. In contrast, melanin is in the epidermis, which occupies the top, up to about 0.1 mm in depth.

Melanin is a natural skin pigment. Hair, skin, and eye color in people and animals mostly depends on the type and amount of melanin they have. Melanin is responsible for the particular skin color of the individual. In humans, melanin exists in skin as eumelanin (which is subdivided further into black and brown forms), and pheomelanin. Both absorb light in the range of about 450-about 700 nm wavelength range and have identical or nearly identical (light) absorption coefficients. Eumelanin and pheomelanin are produced in various amounts in the basal layer of the epidermis within cells called melanocytes.

The volume fraction of melanin in the epidermis is about 1 to about 3% for light-skinned individuals, about 11 to about 16% for medium complexion individuals, and about 18—about 43% for individuals with relatively dark complexions. From an optics point of view, epidermal melanin absorption coefficients measured at 694 nm of light wavelength are approximately 2.5/cm for individuals with relatively dark complexions, 0.3/cm for light-skinned individuals, and 1.2/cm for medium complexion individuals.

Hemoglobin exists in the blood in the form of oxyhemoglobin (with oxygen molecules attached to it) and deoxyhemoglobin (without the oxygen molecules attached to it). Oxyhemoglobin carries oxygen in the blood, from the lungs to the tissues, and circulates back to the lungs as deoxyhemoglobin after unloading the oxygen at the tissue level. Oxyhemoglobin absorption peaks at 525 nm and 575 nm of light wavelengths whereas deoxyhemoglobin absorption peaks at 550 nm of light wavelength.

Melanin absorbs light from about 450 nm-about 700 nm and can interfere with spectral measurements of hemoglobin (both oxy and deoxy components). However, the absorption of “red” ranges of wavelength (in the range of about 600-about 700 nm) light is dominated by melanin as compared to the hemoglobin. Hemoglobin absorbs blue, green, and yellow light well, while absorbing red light relatively poorly.

This disclosure is also directed to various optical insulations that can be used in conjunction with the disclosed devices. Optical insulation can be used for shielding various devices to reduce or prevent leakage of electromagnetic radiation and/or light from inside the device exiting the device, and vice versa. This insulation increases accuracy of reflected or transmitted light.

Many medical devices have a typical structure of a Probe and a Monitor. Some devices are dependent on electromagnetic radiation and/or light of visible or invisible spectrum. In these devices, some form of optical shielding or insulation is typical to prevent leakage of electromagnetic radiations in either direction (outside or inside the device probe).

Various materials are used in this context ranging from carbon compounds, rubber compounds, and metals, etc. However, these typical components have drawbacks. Carbon compounds may alter the reflected light, metals coated with plastics are expensive and add to the thickness. Rubber compounds are not suitable as tiny components in the device system. Thus, there is an unmet need for a reliable optical shield and method of using an optical shield method to improve the accuracy of non-invasive devices to measure various blood and/or skin components.

What is desired is a non-invasive system, devices, and method to measure various blood and/or skin components, which can include an optional optical shield. Embodiments of the present disclosure provide devices and methods that address the above needs.

### SUMMARY OF THE DISCLOSURE

The present disclosure is directed to the measurement of blood and/or skin components. The advantages of this technology are a portable, hand-held device (as described below) to measure the blood and/or skin components concentration in real time, and a method for continuous (or nearly continuous) non-invasive blood and/or skin components monitoring.

### BRIEF DESCRIPTION OF THE DRAWINGS

The present disclosure will be better understood by reference to the following drawings, which are provided as illustrative of certain embodiments of the subject application, and not meant to limit the scope of the present disclosure.

FIG. 1 is a graphical illustration of a device of the present disclosure.

FIG. 2A is a graphical illustration of an embodiment of a probe of the device of the present disclosure.

FIG. 2B is a graphical illustration of an embodiment of a probe of the device of the present disclosure.

FIG. 2C is a cross sectional view of an optical shield.

FIG. 3 is a graphical illustration of a probe of the device and a housing of the present disclosure.

FIG. 4 is a graphical illustration of a probe near a human mammal's skin surface.

FIG. 5 is a graphical illustration of a probe near a human mammal's skin surface.

FIG. 6 is a graph of the reflection of light level as compared to hemoglobin level.

FIG. 7 is a graph of the reflection of light level as compared to hemoglobin level.

FIG. 8 is a graph of the reflection of light level as compared to hemoglobin level.

FIG. 9 is a graph of light signal measurements over four different time points for four different areas of a human mammal's skin surface in the same mammal.

FIG. 10 is a graph of absorption percentage over varying wavelengths for two types of hemoglobin: fetal hemoglobin (Hb-F) and adult hemoglobin (Hb-A).

FIG. 11 is a graph of the reflection of light level as compared to hemoglobin level.

FIG. 12 is a graph of the R value as compared to various skin colors; and

FIG. 13 is a graph of E1 to E2 ratio over time.

FIG. 14 is a graph illustrating the measured ratio Z [Blue light] and E1, E2 and total E[Green light] in four subjects with different hemoglobin and different skin tones, and with relatively normal serum bilirubin levels.

FIG. 15 is a graph illustrating a blue light ratio Z and serum bilirubin levels

FIG. 16 is a graph illustrating a direct linear correlation of E/Z with serum bilirubin irrespective of VLS skin tone and with different hemoglobin values.

FIG. 17 is a graph illustrating extrapolation of bilirubin values in two subjects with different skin tones and hemoglobin values.

FIG. 18 is a graph illustrating the reflection of red and green light in three subjects with substantially the same VLS Scale.

FIG. 19 is a graph illustrating the reflection of red and green light in three subjects with substantially the same hemoglobin but with different VLS Scale.

FIG. 20 is a graph illustrating extrapolation of the reflection of red-light level as compared to VLS Scale-melanin level.

### DETAILED DESCRIPTION OF THE DISCLOSURE

In the discussion and claims herein, the term “about” indicates that the value listed may be somewhat altered, as long as the alteration does not result in nonconformance of the process or device. For example, for some elements the term “about” can refer to a variation of ±0.1%, for other elements, the term “about” can refer to a variation of ±1% or ±10%, or any point therein.

As used herein, the term “substantially”, or “substantial”, is a broad term and is used in its ordinary sense, including, without limitation, being largely but not necessarily wholly that which is specified, which is equally applicable when used in a negative connotation to refer to the complete or near complete lack of an action, characteristic, property, state, structure, item, or result. For example, a surface that is “substantially” flat would mean either completely flat, or so nearly flat that the effect would be the same as if it were completely flat.

As used herein terms such as “a”, “an” and “the” are not intended to refer to only a singular entity but include the general class of which a specific example may be used for illustration.

As used herein, terms defined in the singular are intended to include those terms defined in the plural and vice versa.

References in the specification to “one embodiment”, “certain embodiments”, some embodiments” or “an embodiment”, indicate that the embodiment(s) described may include a particular feature or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper”, “lower”, “right”, “left”, “vertical”, “horizontal”, “top”, “bottom”, and derivatives thereof shall relate to the invention, as it is oriented in the drawing figures. The terms “overlying”, “atop”, “positioned on” or “positioned atop” means that a first element, is present on a second element, wherein intervening elements interface between the first element and the second element. The term “direct contact” or “attached to” means that a first element and a second element are connected without any intermediary element at the interface of the two elements.

Reference herein to any numerical range expressly includes each numerical value (including fractional numbers and whole numbers) encompassed by that range. To illustrate, reference herein to a range of “at least 50” or “at least about 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” or “less than about 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc.

The present disclosure is directed to devices and methods of measuring various blood components, such as different forms of hemoglobin, such as oxy-hemoglobin (O-Hb) and deoxy-hemoglobin (d-Hb).

A hemoglobin molecule has two units, Heme and Globin. The Heme part has one iron as Fe²⁺ or Ferrous form and Pyrrole rings. Globin is tetrameric or with four amino acid chains. These chains are Alpha, Beta, Gamma and Delta globin chains. Hemoglobin therefore has a Heme and Four Globin Chains. For example, Hb-A has two alpha and two beta chains. Table 1 below summarizes the three types of Hemoglobins in health.

TABLE 1

| | | | |
|---|---|---|---|
| Hemoglobin-A | Heme + α2β2 | Hb-A | 98 to 98.5% of |
| | | | total hemoglobin |
| Hemoglobin-A2 | Heme + α2δ2 | Hb-A2 | 1 to 1.5% of |
| | | | total hemoglobin |
| Hemoglobin-F | Heme + α2γ2 | Hb-F | In Newborns till |
| | | | around 8 months |

Oxy-hemoglobin refers to the amount of hemoglobin having oxygen bound to the heme component, while deoxy-hemoglobin refers to the amount of hemoglobin not having bound oxygen. Oxy-hemoglobin and total hemoglobin retain a ratio based on health. When hemoglobin is broken down, globin chains are added to the amino acid pool. Heme is split and all the Pyrrole rings open. These are later metabolized into Bilirubin. When the concentrations are high, more light is being absorbed. Hence, by measuring reflected light, as discussed further below, the concentration of oxy-hemoglobin, and deoxy-hemoglobin can be determined.

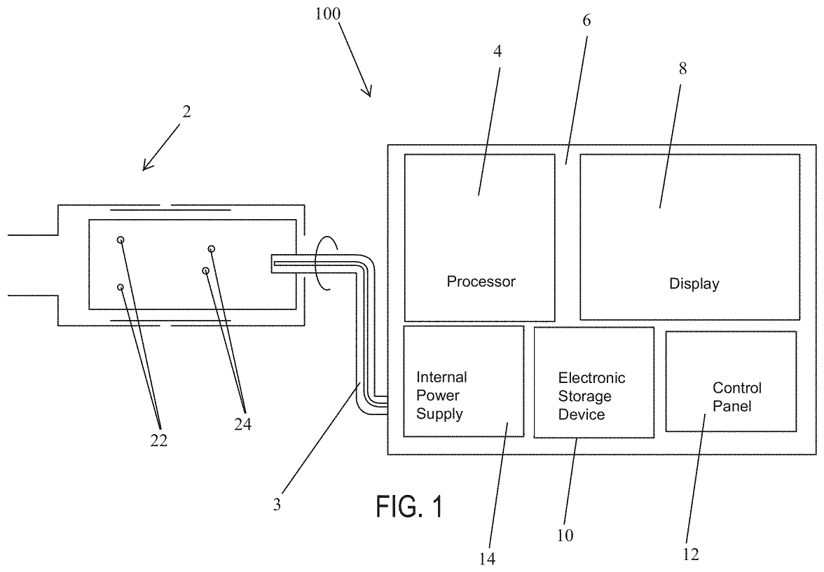

Based on the above a probe device can be utilized to measure reflected light to determine a ratio of oxy-hemoglobin to deoxy-hemoglobin. For example, a probe device 100 is shown in FIG. 1. Oxy-hemoglobin (O-Hb) and deoxy-hemoglobin (d-Hb) have unique spectroscopic properties as compared to each other. O-Hb has an absorption peak between about 515 nm to about 535 nm, between about 520 nm to about 530 nm, or about 525 nm. d-Hb has an absorption peak at between about 540 nm to about 560 nm, between about 545 nm to about 555 nm, or about 550 nm.

The device 100 can include a probe (device) 2 and a processor 4. As used herein, the term “processor” may refer to, is part of, or includes circuitry capable of sequentially and automatically carrying out a sequence of arithmetic or logical operations; recording, storing, and/or transferring digital data. The term “processor” may refer to one or more application processors, one or more baseband processors, a physical central processing unit (CPU), a single or multiple-core processor, and/or any other device capable of executing or otherwise operating computer-executable instructions, such as program code, software modules, and/or functional processes.

In this embodiment the probe 2 is connected to the processor 4 through a suitable cable 3, which is configured to transmit electrical signals. However, in other embodiments, the probe 2 can wirelessly communicate with the processor 4 through any suitable wireless protocol, including but not limited to Wi-Fi, Bluetooth®, Near Field Communication (NFC), etc. In yet other embodiments, the processor 4 can be within the probe 2 itself

The processor 4 can be included in a housing 6. The housing 6 can also include an electronic storage device 10. As used herein, the term “electronic storage device” includes any type of integrated circuit, microcontroller and/or other storage device adapted for storing digital data including, without limitation, ROM, PROM, EEPROM, DRAM, SDRAM, DDR/2 SDRAM, EDO/FPMS, RLDRAM, SRAM, “flash” memory (e.g., NAND/NOR), 3D memory, and PSRAM.

The housing 6 can also include a display 8. The display 8 can be any suitable display, such as a liquid crystal display (LCD), a cathode ray tube display, a light emitting diode (LED) display, or the like, which can display various information determined by the probe 2 and/or stored within the memory 10. Optionally, the display 8 can also be an input by receiving touch input from a user on or near a portion of the display 8. Alternatively, or in addition to, the display 8 being an input, the housing 6 can also include a control panel 12, which can accept various inputs from a user. These inputs are described in more detail below.

Optionally, in this embodiment, the housing 6 can also include an internal power supply 14, such as a battery. However, in other embodiments, the probe 2 and/or processor 4 can receive power from an external source. In yet other embodiments, the probe 2 itself can include a power supply 14.

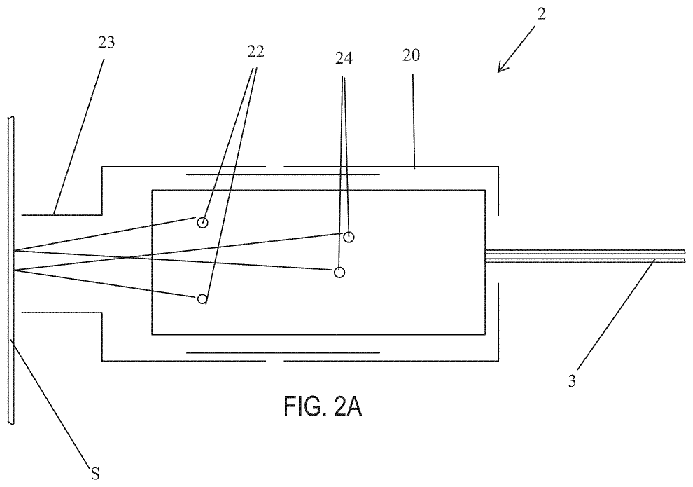

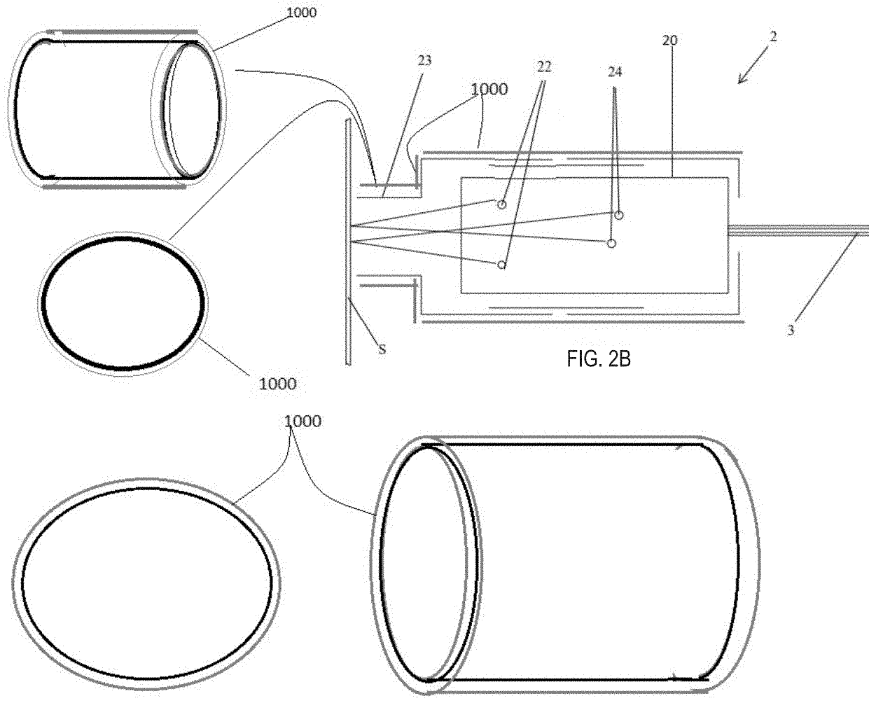

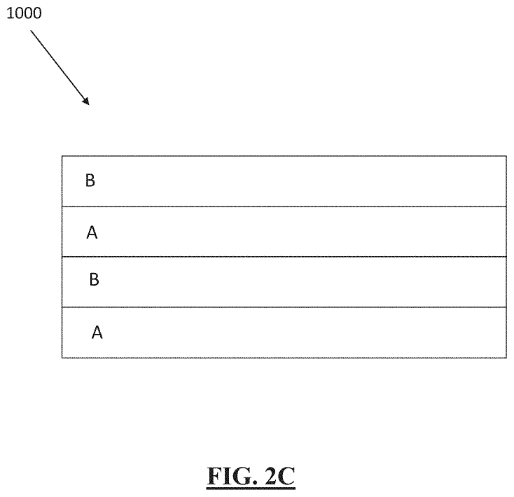

The probe 2 is illustrated in more detail in FIG. 2A, including a nozzle 23 of the probe 2. The nozzle 23 can be any suitable, hollow, or tubular structure that can be formed to be any suitable length to allow for accurate measurement of reflected light by the probe 2.

In FIG. 2A, within the probe housing 20 are a plurality of light emitting diodes 22. The wavelengths of each of the plurality of these light emitting diodes 22 can be the same or different, can be fixed or variable, and can be any suitable wavelength, in any suitable range, such as about 450 nm to about 580 nm. Specific examples of such wavelengths include, but are not limited to about 525 nm, about, 545 nm, about 550 nm, and about 575 nm. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more.

The plurality of light emitting diodes 22 can be activated individually, or in any suitable sequence, to illuminate a surface, such as a skin surface (any suitable portion of the epidermis) of a mammal.

Upon illumination from the light emitting diodes 22, one or more sensors 24 (two are shown in the figures for illustrative purposes), which are configured to sense an amount of light, for example a photodiode, do measure the light reflected from the surface. These one or more sensors 24 then convert that measured light to a suitable electrical signal. In the present disclosure, the light emitting diodes 22 can emit light at any given interval, from about 0.1 second or less, to about 0.5 seconds or more, about 1 second or more, about 5 seconds or more, about 30 seconds or more, about 1 minute or more, about 2 minutes or more, about 5 minutes or more, or about 10 minutes or more. Accordingly, the one or more sensors 24 can be configured to sense the amount of reflected light at times corresponding to whatever time period is selected for light emission.

The probe 2 of FIG. 2A can also include optical insulation in and/or on one or more portions of the probe 2, as illustrated in FIG. 2B.

As shown in FIG. 2B an optical shield 1000 can be included on any surface, internal and/or external of the nozzle 23. Additionally, the optical shield 1000 can be included on any surface, internal and/or external of the probe housing 20.

The optical shield 1000 can be operably attached to any portion of the probe 2 in any suitable way, such as through an adhesive and/or a mechanical connection such as e.g. a clip, a connector, a staple, a fastener, etc.

The optical shield 1000 can comprise at least two alternating layers. For Example layer “A” operably attached to layer “B”. As another example, a first layer “A” operably attached to a first layer “B”, with the first layer “B” operably attached to a second layer “A”. As another example, first layer “A” operably attached to first layer “B”, with the first layer “B” operably attached to a second layer “A”, with the second layer “A” operably attached to a second layer “B”, and so on for tens or hundreds of layers. This example of a four-layer optical shield 1000 is shown in FIG. 2C, which is a cross sectional view of the optical shield 1000. Although it is described as “A” being the initial layer, in other embodiments, the initial layer can be a first “B” layer with a first “A” layer operably attached to the first “B” layer.

Layer “A” can be formed of any suitable foil material. As used herein, the term “foil” refers to a general metal layer, a sheet of metal, plated metal, sputtered metal or metal layer formed or deposited in any fashion known to one of skill in the art. Such foils may be comprised of, for example, aluminum, cobalt, titanium, cadmium, chromium, magnesium, tungsten, platinum, gold, silver, lead, tin, zinc, iron, copper, manganese, nickel, combinations thereof, and/or alloys thereof.

Layer “B” can be formed of any suitable sheet material, such as a woven sheet and/or a nonwoven sheet. The woven sheet or nonwoven sheet may be comprised of a paper material. As used herein the term “paper” means a fibrous material characterized by a plurality of discrete fibers. The fibers can be plant or animal derived, synthetic, or some combination of these. The fibers can be virgin material, recycled material, or a combination thereof. In “plant-derived fibrous materials” the filaments are at least predominantly of plant origin, examples of which include wood, bamboo, sugar cane, wheat straw, grass, reed grass, coconut fiber, hemp fiber, jute palm, jute, papyrus, papyrus, rice, ficus, mulberry, fibers, cotton, yucca, sisal, bowstring hemp and New Zealand flax. As used herein, a “nonwoven sheet” refers to a manufactured sheet, web, or batt of directionally or randomly orientated fibers, bonded by friction, and/or cohesion, and/or adhesion. The fibers may be of natural or man-made origin and may be staple or continuous filaments or be formed in situ. Commercially available fibers may have diameters ranging from less than about 0.001 mm to more than about 0.2 mm and may come in several different forms such as short fibers (known as staple, or chopped), continuous single fibers (filaments or monofilaments), untwisted bundles of continuous filaments (tow), and twisted bundles of continuous filaments (yam).

As used herein, the term “woven sheet” refers to a material containing a structure of fibers, filaments or yarns, which are orderly arranged in an interengaged fashion, woven fabrics typically contain interengaged fibers in a “warp” and “fill” direction. The warp direction corresponds to the length of the fabric while the fill direction corresponds to the width of the fabric. Woven fabrics can be made on a variety of looms including, but not limited to, shuttle looms, Rapier looms, projectile looms, air jet looms and water jet looms. The woven sheet can comprise any suitable material, as natural and/or synthetic materials, and the woven sheet can comprise virgin material, recycled material, or a combination thereof.

Each layer “A” and layer “B” can be joined to one another in any suitable way, such as through an adhesive and/or a mechanical connection such as e.g. a clip, a connector, a staple, a fastener, etc.

Each layer “A” and layer “B” can be the same thickness or a different thickness, with layer “A” being any suitable thickness, such as about 0.001 mm to about 1 mm, or about 0.01 mm to about 0.5 mm, or about 0.02 mm to about 0.1 mm, or about 0.025 mm, and layer “B” being any suitable thickness, such as about 0.001 mm to about 2 mm, or about 0.01 mm to about 0.5 mm, or about 0.05 mm to about 0.2 mm, or about 0.1 mm.

The optical shield 1000 can prevent or reduce the amount of electromagnetic radiation and/or light that is generated within the probe 2 from emitting from any portion of the probe 2 that it is not intended to be emitted from (e.g. any portion other than through the nozzle 23). This can be effected by operably attaching the optical shield to any internal and/or external surface of the probe 2.

Also, the optical shield 1000 can prevent or reduce the amount of electromagnetic radiation and/or light that is generated outside of the probe 2 from entering into any portion of the probe 2 that it is not intended to be received into (e.g., any portion other than through the nozzle 23). This can be effected by operably attaching the optical shield to any internal and/or external surface of the probe 2.

As an example, when the optical shield 1000 is included on an interior surface of the probe 2, when the plurality of light emitting diodes 22 emit light, the light passes through the nozzle 23, towards a mammal's skin, exclusively, or at a very high percentage of emitted light, with little or no light exiting the probe 2 other than through the nozzle 23.

Similarly, as an example, when the optical shield 1000 is included on an interior surface of the probe 2, when the one or more sensors 24 detect light, the light detected passes through the nozzle 23, reflected from a mammal's skin, exclusively, or at a very high percentage of reflected light, with little or no light entering the probe 2 other than through the nozzle 23.

Although the optical shield 1000 is described as being utilized with probe 2, in other embodiments, the optical shield 1000 can be included in and/or on any device that emits light and/or electronic radiation and can also be included in and/or on any device that detects light and/or electronic radiation.

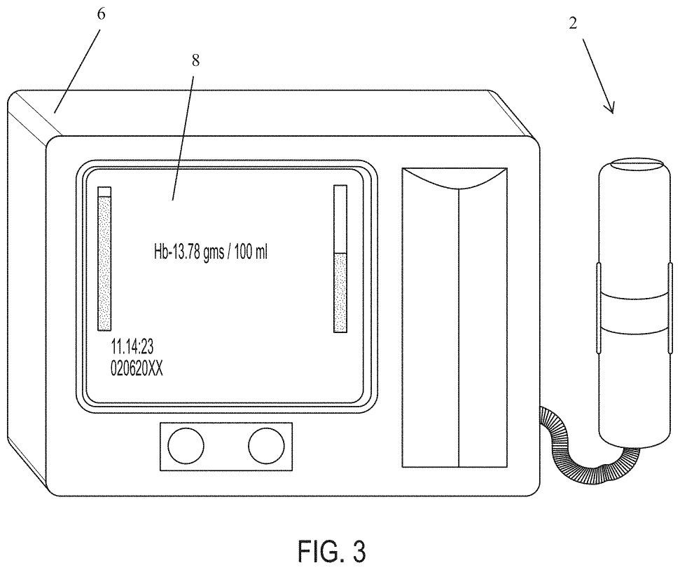

FIG. 3 is one embodiment of a probe 2 connected to a housing 6, with the housing 6 including the display 8 and various inputs and controls. The opening at the vertical upper portion of the probe 2 is where each light is transmitted (from the plurality of light emitting diodes 22) and collected (by the one or more sensors 24) through. This opening can be a void, or can include a substantially transparent barrier, such as a plastic and/or glass barrier.

The display 8, which can optionally be used as an input, can be used to display various data, such as the name of the mammal the probe 2 is to be applied to, date, time, status of the probe 2, power indicator, total hemoglobin, oxy-hemoglobin level, deoxy-hemoglobin level, and/or a ratio of oxy-hemoglobin to deoxy-hemoglobin.

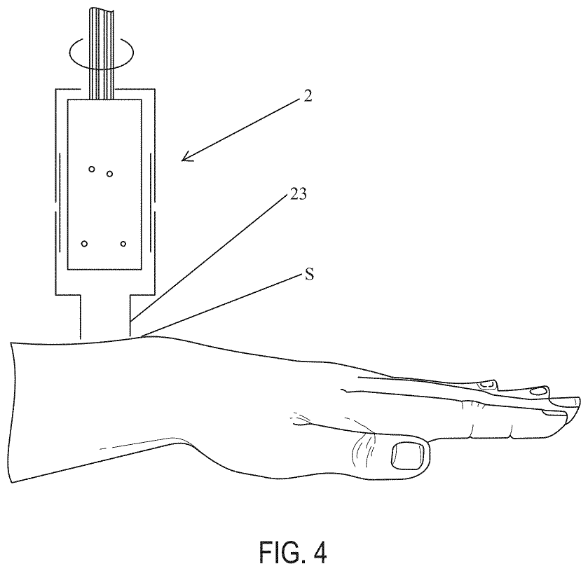

The probe 2 can be placed into contact with any portion of a mammal's skin (S), for example a human's wrist and/or hand as shown in FIG. 4. In other embodiments, the probe 2 can be placed into contact with any portion of a mammal's face, head and/or neck. As can be seen in FIG. 4, the nozzle 23 of the probe 2 is placed near or in contact with the portion of the human's wrist and is held there by another user (or the human themselves, or by a wearable structure). This wearable structure can be a watch and/or bracelet type structure (or any suitable band structure that extend around any digit and/or limb (including a neck) of a mammal) that includes components of the probe 2, as well as one, a plurality or all other components of the probe device 100 (either internally in the pulse oximeter or connected through any suitable wired and/or wireless connection). This wearable structure can also be a pulse oximeter that includes components of the probe 2, as well as one, a plurality or all other components of the probe device 100 (either internally in the pulse oximeter or connected through any suitable wired and/or wireless connection).

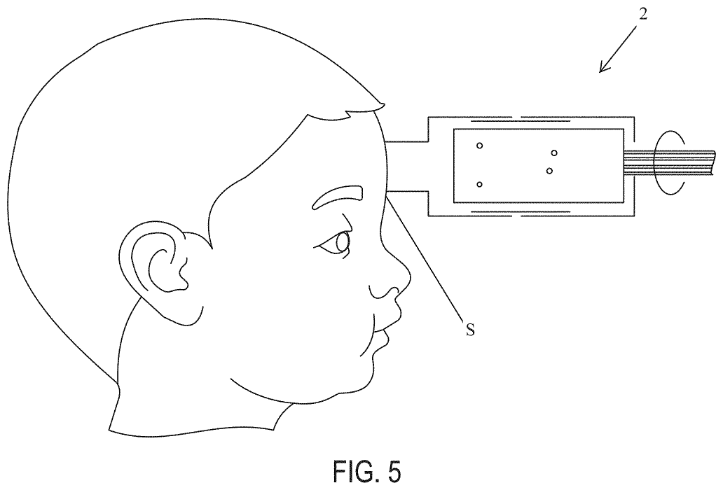

In another embodiment, the probe can be placed into contact with a human's forehead, as seen in FIG. 5.

Alternatively, to the probe of FIGS. 4 and 5, the light emitting diodes 22 and one or more sensors 24 can be included in a wearable structure, for example, similarly to a watch. This wearable structure can also include the processor 4, or the wearable structure can be configured to transmit data to the processor 4 that is external to the wearable structure.

Regardless of probe 2 structure, during operation of the disclosed device in determining oxy-hemoglobin level, deoxy-hemoglobin level, and/or a ratio of oxy-hemoglobin to deoxy-hemoglobin, the light of the plurality of light emitting diodes 22 is directed towards a portion of the mammal's skin.

If that mammal has a relatively low hemoglobin level, a greater proportion of the light emitted from the light emitting diodes 22 is reflected back and received by the one or more sensors 24. For example, more light emitted from the light emitting diodes 22 passes through the epidermis and reflects off of both the upper vascular plexus and the lower vascular plexus, and then back again through the epidermis. Thus, in this example, less light is absorbed in the dermal papillae between the lower vascular plexus and the upper vascular plexus, and between the upper vascular plexus and the epidermis.

In contrast, if that mammal has a relatively high hemoglobin level, a lesser proportion of the light emitted from the light emitting diodes 22 is reflected back and received by the one or more sensors 24. For example, less light emitted from the light emitting diodes 22 passes through the epidermis and reflects off of both the upper vascular plexus or lower vascular plexus, and then back again through the epidermis. Thus, in this example, more light is absorbed in the dermal papillae between the lower vascular plexus and the upper vascular plexus, and between the upper vascular plexus and the epidermis.

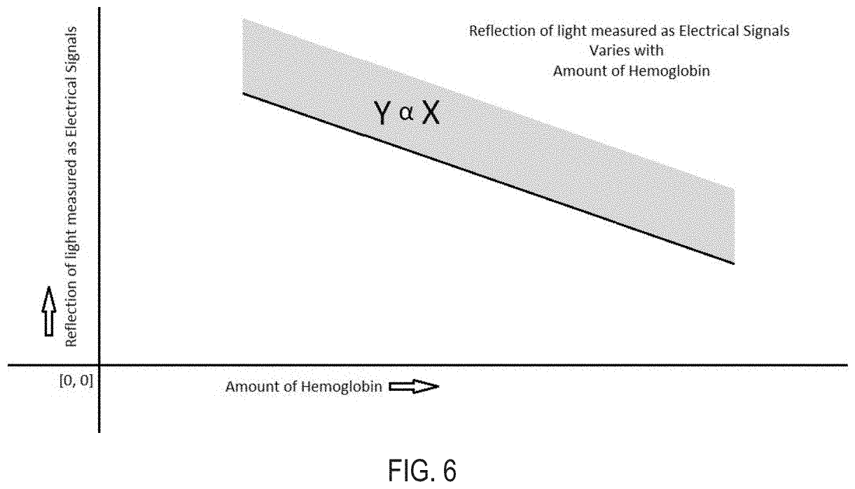

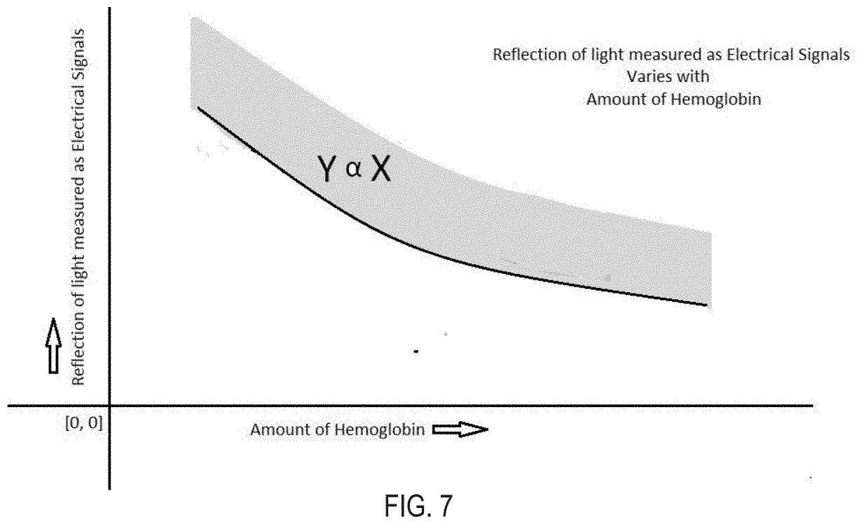

The relationship between the reflection of light and hemoglobin levels is discussed below in reference to FIGS. 6 and 7.

The relationship between the amount of reflection of light measured as an electrical signal (current) (on the Y-axis) and amount of hemoglobin in the blood (on the X-axis) is shown in FIGS. 6 and 7. These graphs are based on several readings from mammals, humans specifically, with different skin color. Mammals with a lighter skin have less melanin in their skin, as compared to mammals with darker skin. In FIG. 6 skin color is kept constant, so that the relationship between electrical signal and the hemoglobin concentration is linear.

Specifically in FIG. 6, the graph illustrates the relationship between the amount of reflection of light measured as an electrical signal (current) (on the Y-axis) by the one or more sensors 24 and amount of hemoglobin in the blood (on the X-axis) of the mammal. The relationship is substantially linear as indicated by the straight line, which applies under the assumption that melanin concentration in the skin of the mammal (or the skin of a plurality of mammals) is substantially constant. The grey shaded area above the substantially straight line represents the symmetrical vertical shift in this linear relationship depending on the skin melanin concentration.

In partial contrast to FIG. 6, as seen in FIG. 7 a graph illustrates the relationship between the amount of reflection of light measured as an electrical signal (current) (on the Y-axis) by the one or more sensors 24 and amount of hemoglobin in the blood (on the X-axis) of the mammal. The line in FIG. 7 is substantially logarithmic due the varying melanin levels of the skin of the humans measured to arrive at the data. The grey shaded area above represents the vertical shift in this relationship depending on the variability in the skin melanin concentration of the mammal under measurement.

The difference between FIG. 6 and FIG. 7 demonstrates the impact melanin content of the mammal's skin can have on the accuracy of a hemoglobin reading.

As a further demonstration of the effect melanin has on hemoglobin determinations, data is presented and discussed with reference to FIGS. 7-10.

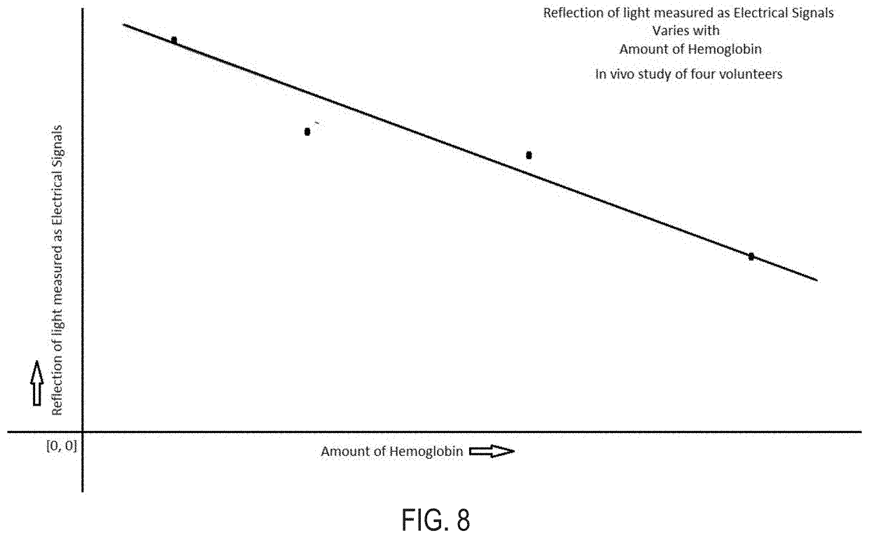

FIG. 8 is the graphical representation of data from four different individuals, with similar melanin content (as measured on the Felix Von Luschan (VLS) skin color chart), using the probe 2 on the same site in each individual (back of the wrist). The VLS scale provides a correlation of the color grade of a human's skin (from 1-36) to the estimated melanin content of that human's skin. Thus, the VLS scale can be relied upon to provide a melanin content substantially accurately for a human based on their assignment to one of 36 skin colors.

Referring again to FIG. 8, the amount of reflection of light measured as an electrical signal (current) is plotted on the Y-axis, measured by the one or more sensors 24, and amount of hemoglobin (measured using the conventional blood draw method) in the blood of the individual is plotted on the X-axis. The relationship is substantially linear as shown with the substantially straight line. As a comparison, the blood hemoglobin of the four different individuals was also measured using the standard cyanmethemoglobin laboratory technique, as shown by the four data points in FIG. 8. As can be seen, there is a substantially accurate correlation between measured light values translated to hemoglobin content as compared to blood tested hemoglobin levels.

As one way to correct for the influence varying melanin levels has on optical hemoglobin determinations, the probe 2 could be made so that the nozzle 23 has a relatively small opening size. Under this embodiment, the melanin concentration of the subject's skin is finite and defined, and if the same amount of light is passed from the plurality of LEDs 22 to a smaller skin surface are of the mammal, the emitted light will encounter a relatively smaller amount of melanin. Since the concentration of melanin is typically in the microgram range, and the concentration of hemoglobin is typically in the gram range, the impact of varying melanin concentrations on optical hemoglobin determinations can be minimized.

As another way to correct for the influence varying melanin levels has on optical hemoglobin determinations, melanin levels can be considered, as discussed below.

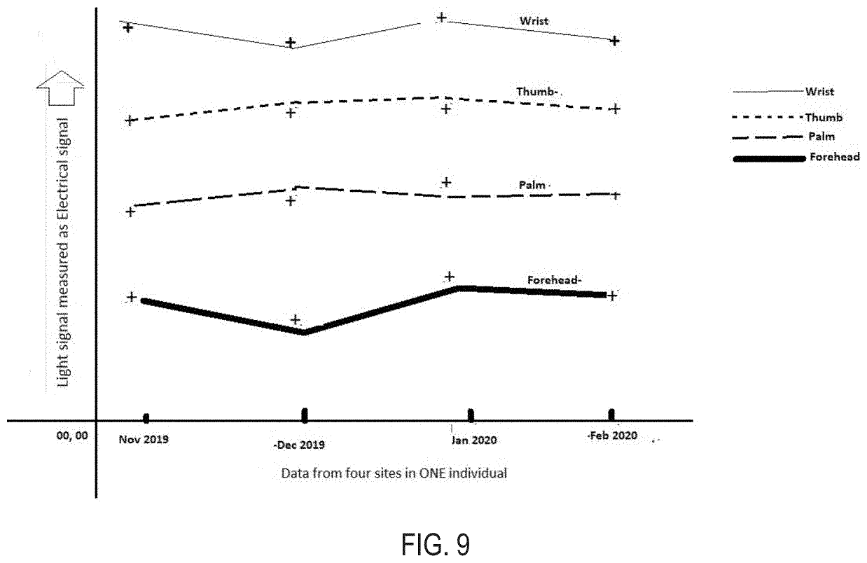

In another example, FIG. 9 is the graphical representation of the temporal and spatial data from one individual, using the probe 2 on four different sites at four different time points, each of which were about four weeks apart. The data points, shown as crosses, are the result of a blood draw and laboratory determination of hemoglobin (substantially constant at 14.4 g/dL).

The amount of reflection of light measured as an electrical signal (current) by the one or more sensors 24 is plotted on the Y-axis and the corresponding time point is plotted on the X-axis. As seen from the graph, the readings are substantially reproducible temporally across 4 different timepoints for the same site. However, there is significant difference across readings obtained from different sites. For example, the amount of reflected light from the wrist is higher and it progressively decreases from wrist-thumb-palm-forehead. Therefore, each probe 2 can be designed for a specific reading location, or the probe 2 and processor 4 can be adjusted to account for the different reading location.

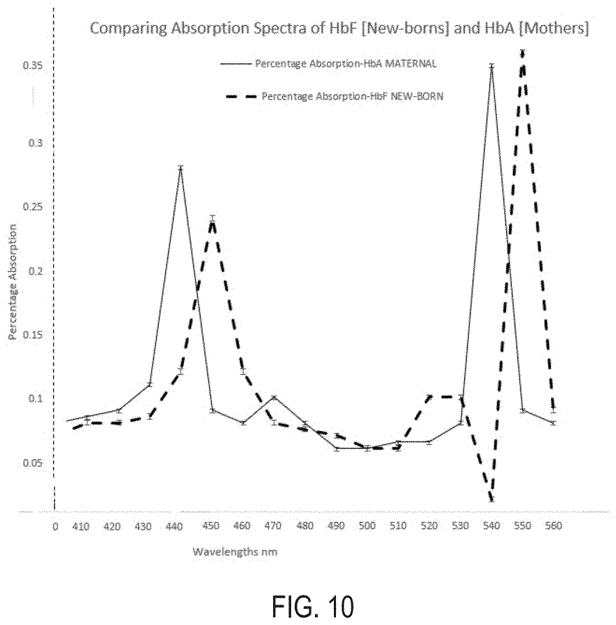

FIG. 10 is the graphical representation of data from 25 mother-infant pairs, to demonstrate similar spectroscopic properties of fetal hemoglobin (Hb-F) and adult hemoglobin (Hb-A), using the disclosed device. Hb-F is the predominant form of the hemoglobin in newborns and is gradually replaced by Hb-A by 8-9 months of age. The percentage of absorbed light is plotted on the Y-axis and the corresponding wavelength (in nanometers) is plotted on the X-axis. Hb-F and Hb-A have remarkably similar spectroscopic properties with the absorption peaks at 450-460 nm and 540-550 nm. Due to similar spectroscopic properties of blood hemoglobin-A and hemoglobin-F, the disclosed device can be used to measure hemoglobin concentration in adults and infants that are younger than 9 months.

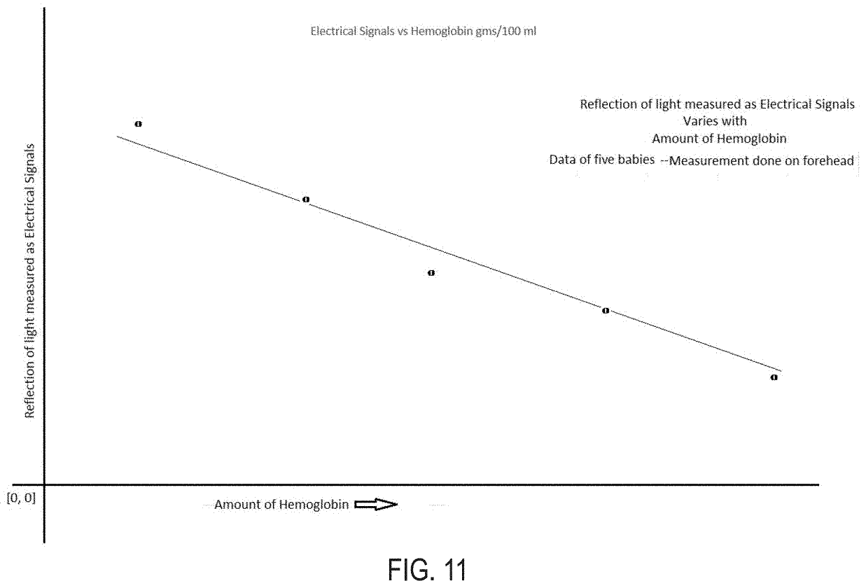

As a further demonstration of this, FIG. 11 is the graphical representation of data from 5 infants, with similar melanin content (as measured on the VLS scale), using the probe 2 on the same site for each infant (forehead). The amount of reflection of light measured as an electrical signal (current) by the one or more sensors 24 is plotted on the Y-axis and amount of hemoglobin in the blood is plotted on the X-axis. The five data points indicated by small circles are the laboratory results of a typical blood draw hemoglobin test. The relationship is substantially linear as shown by the substantially straight line, with the laboratory data being near the linear measurement results of the disclosed device.

Further, Table 2 below includes data from four human subjects, with light brown skin color (VLS scale 24-25). Corresponding R values are calculated using the formula, R=E*H. This E was obtained using the disclosed device, and the blood hemoglobin concentration (H) was obtained using the conventional blood draw method. In Table 2, as well as the rest of the disclosure, the “Ratio” of E1 or E2 is the ratio of light emitted by the plurality of LEDs 22 at that wavelength as compared to the amount detected by the at least one sensor 24 at that wavelength.

TABLE 2

| | | | | Hb (H) | |
|---|---|---|---|---|---|
| | | | Total Ratio | measured by | R |
| Subject | Ratio E1 | Ratio E2 | E = | blood draw | value = |
| no. | (525 nm) | (545 nm) | E1 + E2 | (grams/100 ml) | E*H |
| 1 | 0.692 | 0.671 | 1.363 | 13.71 | 18.69 |
| 2 | 0.736 | 0.749 | 1.485 | 12.29 | 18.25 |
| 3 | 0.633 | 0.616 | 1.249 | 14.56 | 18.19 |
| 4 | 0.650 | 0.626 | 1.276 | 14.32 | 18.27 |

E1 = Ratio of reflected light using the 525 nm LED of the disclosed device
E2 = Ratio of reflected light using the 545 nm LED of the disclosed device

In Table 2 above and throughout the disclosure, the constant ‘R’ is related to the hemoglobin but also to the skin color or skin pigment melanin. The constant R would be a function of the amount of skin melanin and/or hemoglobin in the given subset of race/ethnicity. Therefore, this factor “R” would be different across different races/ethnicities.

R can be further depicted as, R=kM Where ‘M’ represents variable concentration of Melanin in the skin and ‘k’ is the constant factor related to the hemoglobin.

Due to two variables E (either 1 or 2) and M, the nature of the mathematical relationship between E and H could be substantially linear or substantially logarithmic, and the relationship could be depicted using a substantially logarithmic scale (FIG. 7 noted above) as: R=log (E*H)

The value of R (since R=kM) is dependent on the amount of melanin “M” in the skin. If the value of M is kept substantially constant, the value of R would be substantially constant. In other words, the value of R will be substantially constant for the subjects with the same skin melanin concentration (M). In such case, the relationship would tend to be substantially linear, and can be expressed (FIG. 6) as: R=E*H

Because the disclosed device measures E at various wavelengths, the amount of hemoglobin H (in grams/100 ml) in the blood can be calculated by the processor 4, with the formula: H (in grams/100 ml)=R/E

The value of R can be obtained through a melanin concentration determination. As noted above, the value of R is variable depending on the skin melanin concentration and would be constant for a specific melanin concentration. Subjects with lighter skin color (with less melanin, such as Caucasian human subjects) have higher R values, compared to the subjects with the darker skin color (with more melanin, such as African American human subjects), with R being substantially constant for subjects with the same or similar melanin concentrations.

The mean and the standard deviation of the R values obtained in Table 2 were calculated. The mean calculated R value, with the standard deviation (S.D), in this case is (mean=18.35, S.D=0.23). Using a 95% confidence interval (CI) for the data of Table 2 case would be: Mean+/−2 S.D for 95% CI is (17.89-18.81). Therefore, the R value for human subjects with the light skin tone (VLS 24-25) is expected to be around 18.35 and would fall between the intervals of 17.89 to 18.81 for at least 95% of the subjects measured.

Similarly, to Table 2 above, Table 3 below is a representation of data from three human subjects, with dark brown skin color (VLS scale 30-31). Corresponding R values were calculated using the formula, R=E*H. Although only one time point is listed in Table 3 for each of the subjects, in other examples, multiple measurements can be undertaken for each subject. These multiple measurements can span the time ranges noted above, such as continuous or near continuous measurement, up to a measurement every several minutes or more.

TABLE 3

| | | | | Hb(H) | |
|---|---|---|---|---|---|
| | | | Total Ratio | measured by | R |
| Subject | Ratio 1 | Ratio 2 | E = | blood draw. | value = |
| no | (525 nm) | (545 nm) | E1 + E2 | (grams/100 ml) | E*H |
| 1 | 0.360 | 0.368 | 0.728 | 15.71 | 11.43 |
| 2 | 0.396 | 0.395 | 0.791 | 14.60 | 11.55 |
| 3 | 0.382 | 0.367 | 0.749 | 16.30 | 12.21 |

E1 = Ratio of reflected light using the 525 nm LED of the disclosed device
E2 = Ratio of reflected light using the 545 nm LED of the disclosed device

Similarly, to the procedure in Table 2, discussed above, for the data of Table 3, the calculated mean and S.D was (mean=11.73, S.D=0.42). Thus, using a 95% confidence interval (CI) for Table 3, Mean+/−2 S.D for 95% CI is (10.89-12.57). Therefore, the R value for subjects with the dark skin tone (VLS scale 30-31) is expected to be around 11.73 and would fall between the intervals of 10.89 to 12.57 for at least 95% of the subjects measured.

Table 4 is a representation of data from two human subjects. Subject X with light brown skin color (VLS grade 24) and subject Y with dark brown skin color (VLS grade 30).

TABLE 4

| | | | | | | Total | | |
|---|---|---|---|---|---|---|---|---|
| | | | | | R value for | hemoglobin (H, | | |
| | | | | | each skin color | (grams/100 ml) | | |
| | | | | | obtained from | calculated by | Hemoglobin | |
| | | | | | previous | the device using | measured using | Difference |
| | Skin | Ratio E1 | Ratio E2 | Total Ratio | experiments | H = R/E with | blood draw. | (grams/100 ml) |
| Subject | color | (525 nm) | (550 nm) | E = E1 + E2 | with (95% C.I.) | (95% C.I.) | (grams/100 ml) | %) |
| X | Light | 0.668 | 0.672 | 1.340 | 18.35 | 13.69 | 13.90 | (−0.21, 1.5) |
| | | | | | (17.89-18.81) | (13.35-14.0) | | |
| Y | Dark | 0.411 | 0.396 | 0.807 | 11.73 | 14.53 | 14.80 | (−0.27, 1.82) |
| | | | | | (10.89-12.57) | (13.49-15.58) | | |

E1 = Ratio of reflected light using the 525 nm LED of the disclosed device.
E2 = Ratio of reflected light using the 545 nm LED of the disclosed device.

95% C.I.=95% confidence interval obtained by Mean+2 (Standard deviation).

In Table 4, for each subject, values of E were obtained using the disclosed device (column 4). Next, depending upon the skin color, corresponding mean R values (with 95% C.I.) for each skin color were chosen as shown in column 5 (based on Tables 2 and 3 above). Hemoglobin values were calculated using the formula, H=R/E, and are shown in column 6 along with the limits of agreement. These values were compared to those obtained by the conventional blood draw method (column 7).

As shown in column 8, the differences between the hemoglobin values obtained using the disclosed device and conventional blood draw are remarkably small and fall well within the limits of agreement. Specifically, for both subject X and subject Y, the disclosed device was able to accurately estimate the blood hemoglobin value. The calculated values 13.69 (grams/100 ml) (for Subject X) and 14.53 (grams/100 ml) (for Subject Y) are close to the values measured by the conventional blood draw method (13.90 (grams/100 ml) and 14.8 (grams/100 ml) respectively). In both examples, the difference between the device calculated hemoglobin value and the traditional (blood) draw measured value was about 0.2-0.3 (grams/100 ml). This difference is small and is significantly less than the currently available, approved by the Food and Drug Administration (FDA), non-invasive devices.

As a further example, multiple readings were obtained from a number of subjects with different skin colors using the disclosed device on the back of each of their wrists. The R value and mean were calculated for each individual relied on for data in Tables 2 and 3.

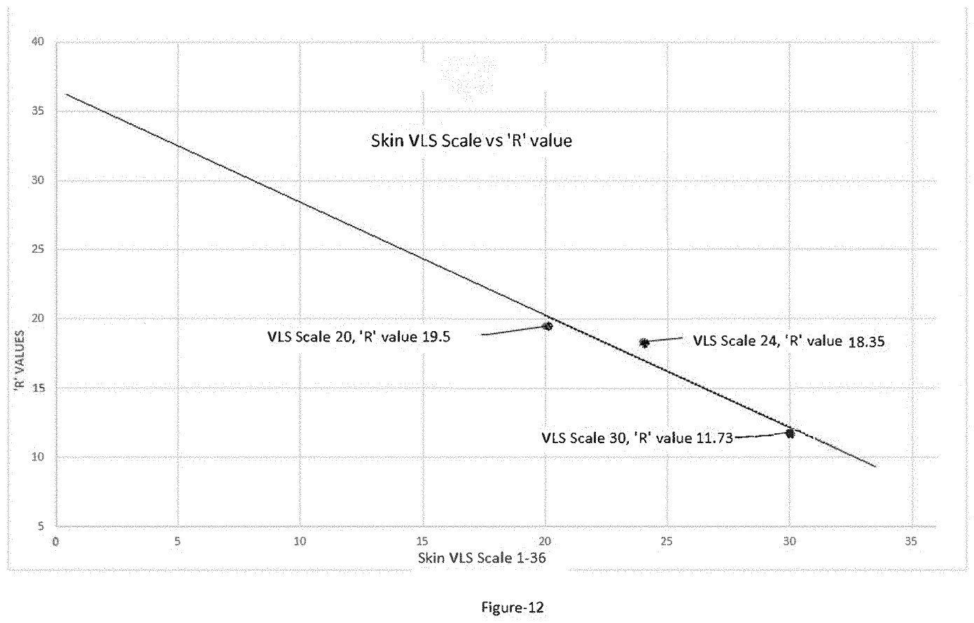

The average R was then plotted on the Y-axis and corresponding skin color (categorical VLS scale from 1-36) on the X-axis. These results are illustrated in FIG. 12. For subjects with light skin color (VLS 20), the mean R value was 19.5. For subjects with slightly darker skin (VLS 24), the mean R value was 18.35. The R value further decreases (mean value 11.73) for subjects with very dark skin (VLS 30). This data demonstrates a relatively consistent, substantially linear inverse relationship between the VLS scales and the R value. These findings support that the R value is significantly lower (around 5-10) in subjects with higher VLS scales as compared (around 35-40) to subjects with lower VLS scales.

Another embodiment of the present disclosure is directed to devices and methods for measuring bilirubin in a mammal, as further discussed in Example 3 herein.

A hemoglobin molecule has two units, Heme and Globin. The Heme part has one iron as Fe′ or Ferrous form and Pyrrole rings. Globin is tetrameric or with four amino acids When Hemoglobin breaks it splits in two parts. The globin part goes to amino acid pool. The heme part is further broken to Iron and pyrrole rings. Bilirubin is produced from this pyrrole part. Due to this structural similarity, a blue color absorption band is shared by both Hb and bilirubin.

Similar to the reflectance principles discussed herein, a bilirubin probe device can be utilized to measure reflected light to determine bilirubin levels in blood.

Bilirubin has several peaks within the light absorption spectrum, however, the following peaks were selected: (1) about 430 nm to about 470 nm, about 440 nm to about 460 nm, about 445 nm to about 455 nm or about 450 nm; (2) about 505 nm to about 545 nm; about 515 nm to about 535 nm, about 520 nm to about 530 nm, or about 525 nm; (3) about 525 nm to about 565 nm, or about 535 nm to about 555 nm, or about 540 nm to about 550 nm, or about 545 nm. However, in other embodiments, the wavelengths can differ from any of the above values by about 0.001%, about 0.01%, about 0.1%, about 0.5%, about 1%, about 2%, about 3%, about 4%, about 5%, about 7%, about 9%, about 10%, about 13%, about 15%, about 20% or more. Thus, one or more light emitting diodes can be used to emit light at one or more of these wavelengths. From the reflectance values of these wavelength ranges, ratios can be used to determine the bilirubin level, such as ratios ‘Z’ and ‘E1 and E2 and total ratio E=[E1+E2] and ‘E/Z’, discussed further herein.

The ratio (1) or “Z” is “blue” light range reflection ratio, with an absorption peak that is shared with bilirubin, hemoglobin and melanin.

The relation is inverse, with k being a constant.

Zα1/B*Hb*M, where B is the amount of bilirubin, Hb is the amount of hemoglobin and M is the amount of melanin

Z=k/B*Hb*M

Reflection ratio (2)+(3) or “E” is a “green” light reflection ratio, with an absorption peak that is shared by hemoglobin and melanin

The relation is inverse, with p being a constant.

Eα1/Hb*M

E=p/Hb*M

E/Z=p/k B*Hb*M/Hb*M

E/Z=p/k B

Or

Ratio E/Z α Bilirubin, since p and k are both constants.

E/Z varies directly with respect to Bilirubin and is irrespective of melanin content in the mammal's skin. Thus, in this example, the E/Z ration is independent of skin tone/color (melanin content).

In this embodiment, directed to bilirubin detection, the probe 2 of FIG. 2A can also be used. In FIG. 2A, within the probe housing 20 are a plurality of light emitting diodes 22. The wavelengths of each of the plurality of these light emitting diodes 22 can be the same or different, can be fixed or variable, and can be any suitable wavelength, in any suitable range, such as (1)-(3) noted above.

As one example, one of the plurality of light emitting diodes 22 can be configured to emit light in the (1) wavelength range, while another one, two or more of the plurality of light emitting diodes 22 can be configured to emit light in the (2) and (3) wavelength ranges. In this example, the light emitting diode configured to emit light in the (1) wavelength range is caused to emit light for a first time period and then stop emitting light. Reflected light from the mammal's skin is received and detected by the one or more sensors 24. This reflected light of the (1) wavelength range is referred to herein as Z [blue light ratio].

In this example, one, two or more of the plurality of light emitting diodes 22 are configured to emit light in the (2) and (3) wavelength ranges, with light emitted in the (2) range for a second time period, after the first time period, and light emitted in the (3) range for a third time period, after the second time period. Reflected light from the mammal's skin is received and detected by the one or more sensors 24 for the (2)(referred to herein as E1) and (3) (referred to herein as E2) wavelength ranges. E1 and E2 can be added together to a total value, E, referred to herein as E or [green light ratio].

Example 3 below discusses use of probe 2 and bilirubin detection in more detail.

Another embodiment of the present disclosure is directed to devices and methods for measuring the skin constituent melanin in a mammal, as further discussed in Example 4 herein.

The present disclosure includes a device and a non-invasive method for calibration and measurement of skin tone/color. Or in other words, a technology to measure and/or assign a skin color grade for mammals of different skin colors. This probe 2 of FIG. 1 can be used to make this assessment, by providing illumination from the one or more of the light emitting diodes 22, with the one or more sensors 24 detecting a reflected signal.

Determination of melanin concentrations is indicated in several clinical situations such as grading the skin tone, measuring the effect and degree of tanning, and to monitor pigmented skin lesions such as melanoma. Current methods to measure melanin in the skin consist of spectrometry of melanin and histological analysis of skin for melanin content. Histological analysis requires an invasive skin biopsy and is not possible in routine and frequent monitoring. Melanin spectroscopy requires additional specialized equipment, and highly trained personnel.

Comparing skin color with Felix Von Luschan Chromatic (VLS) scale is a known clinical method to grade the skin tone. In the VLS scale, skin tone (color) is graded from 1 to 36. Increasing numbers correspond to the increasing melanin contents and darker skin tones. There is a relatively good correlation between the estimated skin tone of the VLS scale according to an operator's visual inspection as compared to measured melanin content using the reflectance spectroscopy.

Though simple to administer, this estimation based on visual inspection suffers from the observer's bias, as the administration relies on the subjective assessment of the skin tone by the examiner's eyes. Additionally, external factors such as ambient illumination could have impact on the skin tone estimation. Thus, there is an unmet need for a reliable, and non-invasive method for determining melanin in the skin.

The one or more light emitting diodes 22 of the probe 2 can be configured to emit light in the “white” light spectrum and can be configured to emit light of any particular power, including but not limited to about 10 mW to about 40 mW, or more. The light emitting diodes 22 of the probe 2 can emit light for any suitable amount of time, such as about 1 second to about 60 seconds, or about 10 seconds to about 20 seconds.

After absorption by the skin's epidermis and/or dermis, the skin reflects a part of light depending on the concentration of melanin and hemoglobin (chromophores) in the skin. The one or more sensors 24 detect the reflected light from skin area and produces an image.

The image(s) detected by the one or more sensors 24 can be analyzed by the processor 4 (or any suitable external processor). The processor 4 can perform qualitative and quantitative analysis for three different colors —red, green, and blue reflected by the skin area. The intensity and average of these colors can be analyzed by the processor 4 to determine a melanin concentration of the epidermis of the subject mammal.

Melanin and hemoglobin (in the skin) absorb visible light from 450 to 700 nm of wavelengths. Hemoglobin absorbs predominantly green light (about 525-about 575 nm). Melanin absorption predominantly occurs in the red light (about 600-about 700 nm) zone. Although both melanin and hemoglobin absorb the visible light in the green-red spectra, there are inherent differences in the relative affinities of melanin and hemoglobin for the red and green light respectively.

Therefore, when the white light (containing red, blue, and green fractions) is emitted by the one or more light emitting diodes 22 onto comparatively darker skin with a relatively high melanin content, a larger fraction of the red light is absorbed in the skin due to higher melanin content. Thus, a smaller amount of red light is reflected from the skin- and therefore a comparatively smaller amount of red light is detected by the one or more sensors 24. Conversely, in case of a mammal with less melanin content in their skin, less melanin in the skin leads to higher amount of reflected red light from the skin, and therefore a comparatively larger amount of red light is detected by the one or more sensors 24.

This detection and analysis is further discussed in Example 4.

### Example 1

In this example, a patient enters a clinical setting. The operator then places the probe 2 on a portion of the patient, for example the patient's wrist.

The one or more sensors 24 detect reflected light, in this example at 525 nm and 545 nm, such that the processor 4 can determine the ratio of both of E1 and E2. The processor 4 makes this determination by determining the ratio of returned light detected by the one or more sensors in comparison to the emitted light from the plurality of LEDs 22, at each wavelength.

The processor 4 then outputs two ratios (E1) and (E2). The processor 4 can then add those values (E1)+(E2) (or a user can manually add those ratios) to determine a total E value. Next, the VLS scale value is determined in one of two ways.

The first way is for a user to estimate the value by a visual inspection and assignment of the patient to a score of 1-36 on the VLS scale. Under this first option, the operator can then manually select the corresponding R value for the selected score (present on a provided chart that includes all VLS scale scores and their corresponding R values). The operator can then manually divide the R value by E.

The second way is for the probe 2 to include an optical sensor (one or more sensors 24, or an additional sensor) that can receive a signal, and the processor 4 can, based on the signal, automatically assign the patient to a VLS scale value (with its corresponding R value), with those VLS scale values and R values being stored in the electronic storage device 10. The processor 4 can then divide the R value by the obtained E value to determine the total hemoglobin value.

### Example 2

In addition to the total hemoglobin as described above in Example 1, the disclosed device can also measure the ratio of oxy-hemoglobin [O-Hb] to deoxy-hemoglobin [d-Hb], as well as change in the ratio, as discussed in this example.

In this example, a patient enters a clinical setting. The operator then places the probe 2 on a portion of the patient, for example the patient's wrists. The total hemoglobin value is then obtained as discussed in Example 1.

Further, since E1 substantially corresponds to the data collected at the 525 nm wavelength, it is indicative of oxy-hemoglobin concentration, while E2, substantially corresponding to data collected at the 550 nm level (in this example, however, about 545 nm level can also be used for data collection), is indicative of deoxy-hemoglobin concentration. The ratio of E1 to E2 can be determined once, or sequentially to monitor the patient's condition.

In this example, blood hemoglobin was measured with the disclosed device for a patient presenting an acute asthma attack. Asthma causes airways to become inflamed and constrict, leading to low oxygen concentration in the blood. The patient had a VLS skin scale of 20, with the corresponding R value of 19.5 based on a value demonstrated by FIG. 12.

At first, the patient had symptoms of breathlessness and cough. The obtained readings are depicted immediately after presentation, at time 2 minutes (Table 5), with the disclosed device. Patient was then observed and treated with medications to relieve cough and breathlessness. However, patient's clinical condition deteriorated. Another set of readings is obtained at 21 minutes with the disclosed device. Patient was subsequently treated with a supplemental oxygen face mask. Patient's condition improved, and a repeat set of observations were obtained after about 20 minutes of oxygen therapy (at 40 minutes), with the disclosed device.

TABLE 5

| | | | | | | Calculated Hb | Measured Hb | Oxygen saturation |
|---|---|---|---|---|---|---|---|---|
| | | | | | R value | using the device. | using the blood | using arterial |
| Time | Ratio E1 | Ratio E2 | Ratio | Total ratio | for skin | H = R/E | draw method. | blood gas |
| (mins) | (525 nm) | (550 nm) | E1/E2 | (E1 + E2) | VLS 20 | (grams/100 ml) | (grams/100 ml) | (normal 97-100%) |
| 2 min | 0.631 | 0.608 | 1.038 | 1.239 | 19.50 | 15.74 | 15.30 | 90% |
| (room air) | | | | | | | | |
| 21 min | 0.652 | 0.588 | 1.109 | 1.240 | 19.50 | 15.73 | 15.40 | 86% |
| (room air) | | | | | | | | |
| 40 min | 0.615 | 0.622 | 0.988 | 1.237 | 19.50 | 15.76 | 15.30 | 96% |
| (Oxygen | | | | | | | | |
| face mask) | | | | | | | | |

E1 = Ratio of reflected light using the 525 nm LED of the disclosed device
E2 = Ratio of reflected light using the 545 nm LED of the disclosed device

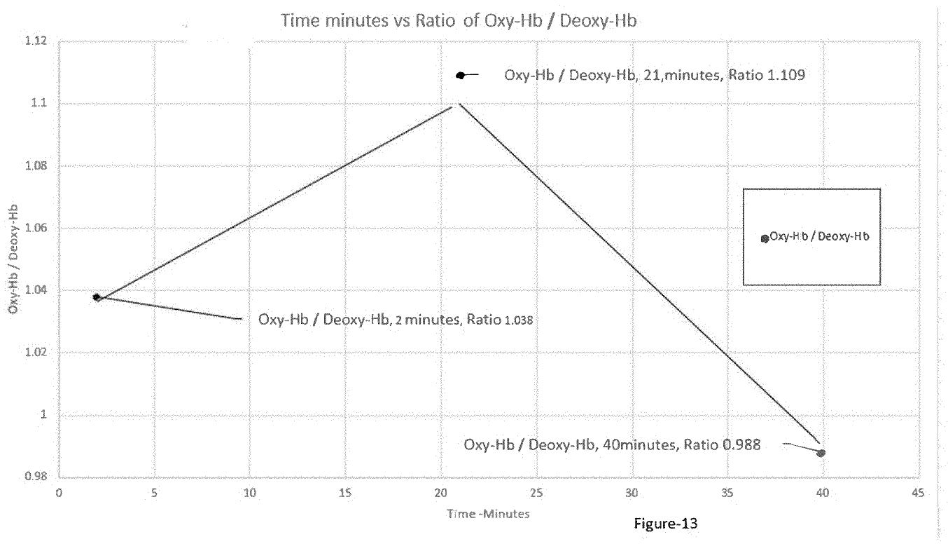

The E1 divided by E2 ratio was obtained at each time point by the processor 4. As seen from Table 5, the ratio increased from 1.038 to 1.109 as patient's clinical condition worsened. After treatment with the supplemental oxygen, patient's condition improved, and the E1/E2 ratio decreased to 0.988. These results are shown in the FIG. 13.

Specifically, in FIG. 13, the ratio of E1/E2 measured using the disclosed device, is plotted on the Y axis, and the corresponding time point (in minutes) is plotted on the X axis. At presentation, the patient had hypoxia (low oxygen content), with E1/E2 ratio 1.038. After 20 minutes, patient's condition and hypoxia worsened, with corresponding increase the ratio to 1.109. Patient was subsequently treated using supplemental oxygen. Patient's hypoxia improved, correlated with the ratio of 0.988.

As seen in FIG. 13, as the ratio of E1/E2 increases, the blood oxygen saturation (an indicator of oxyhemoglobin) decreases. Since E and H have an inverse relationship as previously described (FIG. 6), the ratio E1/E2 is expected to have an inverse relationship with the relative oxy-hemoglobin (O-Hb) concentration. Oxy-hemoglobin is the form of hemoglobin attached to the oxygen molecules and is responsible for the oxygen delivery to the tissue.

Therefore, ratio E1/E2 provides a tool to monitor patient's clinical condition without requiring the need for the invasive arterial blood gas sampling. The ratio can be calculated at the bedside by a user, or the processor 4 could determine this ratio. Currently available non-invasive method (pulse-oximetry) to measure blood oxygen saturation, has limitations in dark-skinned subjects, and during low perfusion states, such as shock. The disclosed device does not have these limitations.

Since the relative ratios of oxy and deoxy hemoglobin are unique for each individual, the absolute value of ratio E1/E2 would vary between individuals. However, the ratio E1/E2 would be specific for the given subject and could be used through serial measurements as a non-invasive means of oxyhemoglobin monitoring. This can significantly decrease the need for the invasive blood collection.

### Example 3

A probe device 2 can be utilized to measure reflected light using diodes with different wavelengths of ranges (1)-(3) noted above, to determine a ratio of Z, E1, E2 and E. Hemoglobin includes a peak in a “blue” wavelength range [420 to 460 nm] and a “green” wavelength range [520-580 nm], thus both bilirubin and hemoglobin include a peak I the “blue” range. This peak of both hemoglobin and bilirubin is due to hemoglobin having a strong Soret band absorption peak at about 420 nm which partially overlaps with the bilirubin absorption peak at about 460 nm.

A subject with a particular VLS scale skin tone has a constant factor R[Hb*E] as discussed in U.S. Pat. No. 11,191,460, the entire contents of which are incorporated by reference. If the subject has low hemoglobin, the subject's E value will increase to compensate for a decrease in Hb. This subject with low hemoglobin will have decreased absorption of light in the “blue” range and comparatively high reflection of light in the “blue” range, with relatively high Z values. The subject's E value is high because the subject's Hb is low. But, a ratio of E/Z will remain substantially constant. The ratio of E/Z is substantially constant irrespective of skin tone and hemoglobin values.

Now if this subject becomes jaundiced and/or has relatively high bilirubin concentrations, then bilirubin will absorb blue light and the subject's ratio Z, reflection of light in the “blue” range, will decrease. Effectively the subject's E/Z ratio will increase corresponding to high bilirubin values.

A subject with a particular VLS scale skin tone has a substantially constant factor R[Hb*E]. The value of E changes inversely with hemoglobin so as keep Hb*E substantially constant. This R is an inverse function of VLS skin tone as discussed in U.S. Pat. No. 11,191,460. Bilirubin has selectively one peak in the “blue” light range. Therefore, this E/Z ratio affected proportionately by an increase in bilirubin does not change with respect to VLS skin tone, Hemoglobin and R values. Thus, the disclosed methods and devices provide accurate measurements of bilirubin irrespective of Hemoglobin values and skin tone.

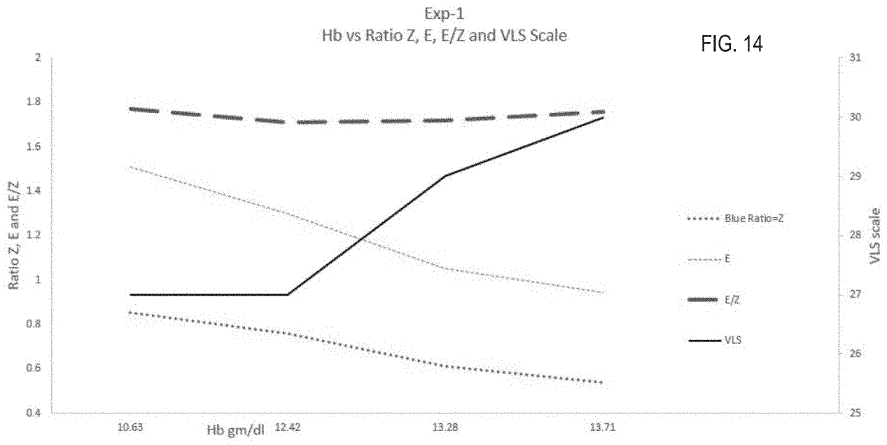

FIGS. 14-17 are different graphs describing the four experiments for non-invasive measurements of Bilirubin

### Experiment-1

Values for Z [“blue” light range], E1, E2 and total E[“green” light range] in four subjects with different hemoglobin and different skin tones but with normal serum bilirubin levels were measured with a device of FIG. 1. Ratio of green light reflections E=E1+E2 and ratio of blue light reflections Z are both functions of hemoglobin and both these ratios change inversely with hemoglobin values. These values also indicate that E/Z is substantially constant irrespective of skin tone and hemoglobin levels.

TABLE 6

| Hb- | Hb-Lab | | | | |
|---|---|---|---|---|---|
| Disclosed | (Blood | Blue | | | |
| Device | Sample) | Ratio = Z | E | E/Z | VLS |
| 10.63 | 10.32 | 0.852 | 1.51 | 1.77230047 | 27 |
| 12.42 | 12.89 | 0.758 | 1.298 | 1.71240106 | 27 |
| 13.28 | 13.81 | 0.612 | 1.053 | 1.72058824 | 29 |
| 13.71 | 13.01 | 0.538 | 0.946 | 1.75836431 | 30 |

The results of Table 6 are shown in FIG. 14.

### Experiment-2

In one subject with jaundice, hemoglobin and bilirubin were measured three times over 10 days. Bilirubin increased, but hemoglobin remained substantially the same. The ratio “E” remained substantially the same over 10 days. Blue light ratio Z however showed an inverse relationship with bilirubin level. The ratio E/Z showed direct linear relationship with serum bilirubin.

TABLE 7

| Disclosed | Lab (Blood | | | |
|---|---|---|---|---|
| Device- | Sample)- | Blue | | |
| Bilirubin | Bilirubin | Ratio = Z | E/Z | E |
| 2.2 | 1.95 | 0.45 | 2.322222222 | 1.045 |
| 3 | 2.97 | 0.39 | 2.651282051 | 1.034 |
| 10 | 10.86 | 0.28 | 3.771428571 | 1.056 |

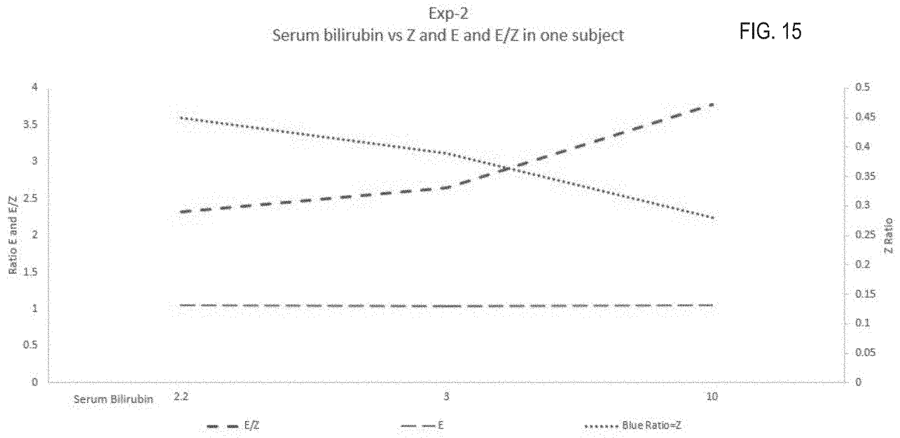

The results of Table 7 are shown in FIG. 15.

### Experiment-3

Bilirubin levels and skin tones [VLS scale], hemoglobin [Hb] and R [Hb*E] and blue light ratio [Z] and E/Z were all measured in three subjects. It was found that serum bilirubin levels as measured in a laboratory blood sample bear little or no correlation with VLS Scale skin tone, Hb, R and E ratio. With different skin tones and different Hb values in these three subjects, ratio Z showed poor correlation with serum bilirubin. But, E/Z showed strong direct linear correlation with bilirubin levels.

TABLE 8

| Bilirubin | Bilirubin | | | | | | |
|---|---|---|---|---|---|---|---|
| assessed by | assessed by | | | | | | |
| Disclosed | Laboratory | | | | R = | | VLS |
| Device | Method | E | E/Z | Hb | E*Hb | Z | Scale |
| 3.65 | 3.28 | 0.9 | 1.77 | 15.4 | 14 | 0.51 | 30 |
| 7.89 | 7.28 | 1.2 | 2.45 | 15 | 18 | 0.49 | 24 |
| 12.67 | 13.21 | 1.43 | 3.17 | 11.25 | 16 | 0.45 | 26 |

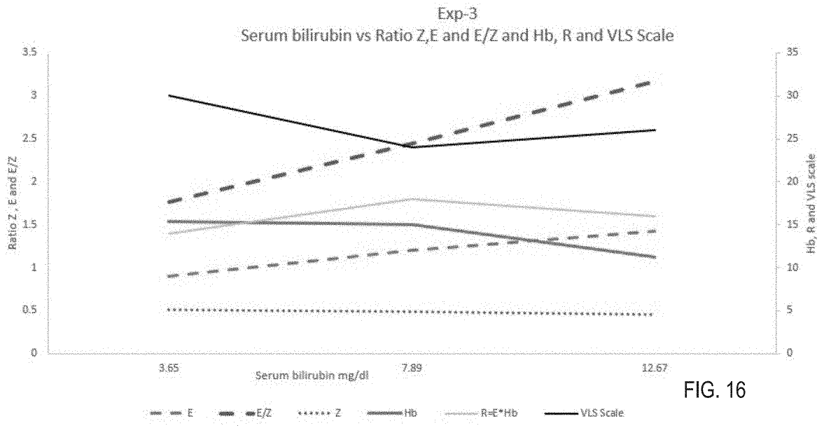

The results of Table 8 are shown in FIG. 16.

### Experiment-4

As shown in experiment-3 that E/Z had strong direct linear correlation with serum bilirubin irrespective of VLS skin tone and with different hemoglobin values, bilirubin values were extrapolated for two subjects with different skin tones and hemoglobin values. E1 and E2 were measured and calculated total as E as discussed in U.S. Pat. No. 11,191,460. Z was measured as well. The E/Z ratio was calculated in these two cases. Using the equation obtained in experiment-3, serum bilirubin was calculated.

E/Z=0.1552*Serum bilirubin+1.2099

rearranging-

[E/Z−1.2099]/0.1552=Serum Bilirubin

TABLE 9

| BILIRUBIN BY | BILIRUBIN IN | | | | | |
|---|---|---|---|---|---|---|
| DISCLOSED | LAB (Blood | | | | | |
| DEVICE | Sample) | E | E/Z | HB | Z | VLS |
| 3.65 | 3.55 | 0.9 | 1.77 | 15.4 | 0.51 | 30 |
| 7.89 | 7.51 | 1.2 | 2.45 | 15 | 0.49 | 24 |
| 12.67 | 12.32 | 1.43 | 3.17 | 11.25 | 0.45 | 26 |
| 16 | 15.56 | 1.5 | 3.7 | 12 | 0.405 | 24 |
| 9 | 9.34 | 1 | 2.605 | 11.5 | 0.38 | 27 |

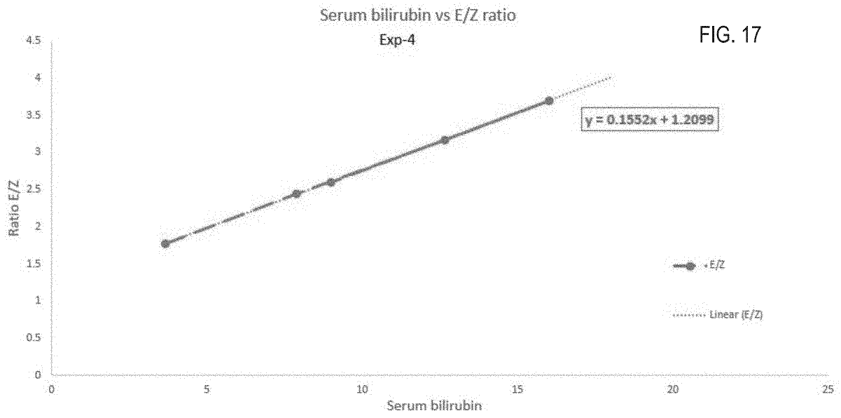

The results of Table 9 are shown in FIG. 17.

### Example 4

This Example 4 discussed skin melanin determination using a probe 2 of the present disclosure.

### Experiment 1

Three subjects with the same or substantially similar VLS skin tone participated in this observational experiment. Using the probe 2, a similar amount of white light was emitted from the one or more light emitting diodes 22 onto the skin surface of each subject. The one or more sensors 24 detected the light reflected from the subjects' skin, which was analyzed by the processor 4 (in this embodiment processor 4, but in other embodiments any processor external to the probe device 100 can perform the analysis) to determine red and green fractions using RGB analysis. The RGB analysis assigns a PIXEL numerical number (from 0-255), depending upon the intensity of the light detected by the one or more sensors 24. For E, a PIXEL value of 10 indicates less intensity of the measured light compared to the PIXEL value of 100.

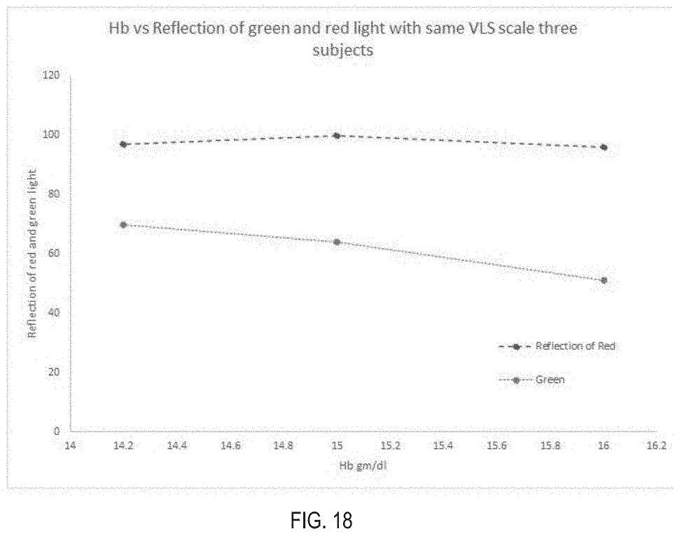

The one or more sensors 24 captured PIXEL intensity (on the Y axis of FIG. 18) for the green and the red-light fractions, which was plotted separately against the hemoglobin values measured using the blood draw method (on the X axis of FIG. 18). The Y axis is a PIXEL intensity value, which is the result of the RGB analysis noted above. This analysis can be performed by the processor 4 (in this embodiment processor 4, but in other embodiments any processor external to the probe device 100 can perform the analysis). The data for FIG. 18 is shown in Table 10 below. There is an inverse relationship between the hemoglobin values and the measured green light fraction value. However, the changing hemoglobin concentration have little to no impact on the red-light fraction value. The impact of melanin is controlled with the same VLS skin tone for all three subjects.

TABLE 10

| Hemoglobin gm/dl | Reflection of | Reflection of |
|---|---|---|
| (Blood Draw) | Red Light | Green Light |
| 16 | 81 | 51 |
| 15 | 83 | 64 |
| 14.2 | 80 | 70 |

### Experiment 2

Three subjects with the same or substantially similar hemoglobin value (measured using the blood draw method) participated in this observational experiment. Using the probe 2, a similar amount of white light was emitted from the one or more light emitting diodes 22 onto the skin surface of each subject. The one or more sensors 24 detected the light reflected from the subjects' skin, which was analyzed by the processor 4 (in this embodiment processor 4, but in other embodiments any processor external to the probe device 100 can perform the analysis) to determine red and green fractions using RGB analysis.

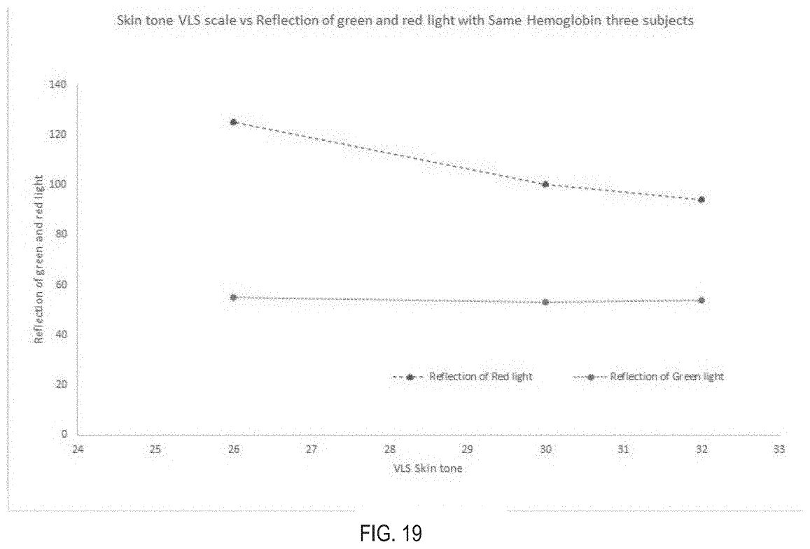

The one or more sensors 24 captured PIXEL intensity (on the Y axis of FIG. 19) for the green (G) and the red (R) light fractions was plotted separately against the VLS skin tone (on the X axis of FIG. 19), with the plotted data presented in Table 11 below. There is an inverse relationship between the VLS skin tone (melanin) and the measured green light fraction value. In addition, there is an inverse relationship between the VLS skin tone (melanin) and the measured red light fraction value. The slope of the red-light faction value is steeper, thus there is a larger melanin-impact on the absorption of the red light. The impact of hemoglobin is controlled with the same (or substantially similar) hemoglobin value for all three subjects.

TABLE 11

| Skin tone | Reflection of | Reflection of |
|---|---|---|
| VLS Scale | Red light | Green light |
| 32 | 80 | 54 |
| 30 | 88 | 53 |
| 26 | 114 | 55 |

### Experiment 3

Based on the experiment 2 of Example 4, the intensity of the red-light fraction value measured using the probe 2 is predominantly affected by the amount of melanin in the skin (as estimated using the validated VLS scale). This value is not significantly affected by varying hemoglobin content, as demonstrated in the first experiment of Example 4. Therefore, the impact of hemoglobin on the red-light fraction is clinically, substantially insignificant. Thus, the changing intensity of the red-light fraction of reflected light, measured using the one or more sensors 24, can be used to inform the VLS skin tone (and melanin) of a given subject.

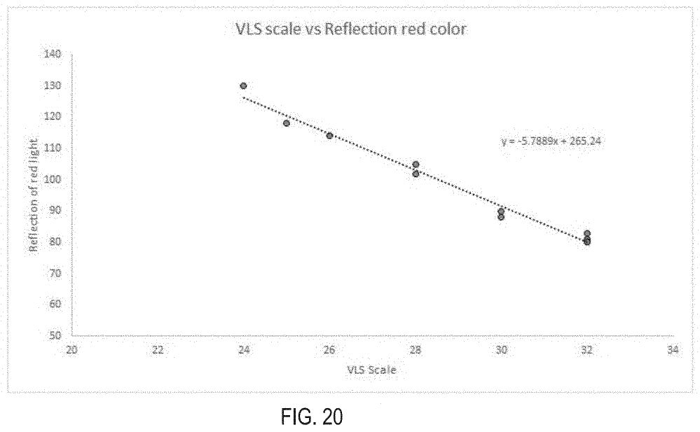

To further substantiate this determination of melanin content, four subjects with varying skin tones participated in this observational experiment. Data from experiments 1 and 2 of Example 4 were pooled. The processor 4 (in this embodiment processor 4, but in other embodiments any processor external to the probe device 100 can perform the analysis) estimated red light faction values on the Y-axis of FIG. 20, which were plotted against the VLS skin tone values (on the X-axis of FIG. 20), with the data points for each provided below in Table 12.

TABLE 12

| VLS Scale | Reflection of red light |
|---|---|
| Participant- 24 | 130 |
| Participant-25 | 118 |
| Participant-28 | 102 |
| Participant-28 | 105 |
| Participant-32 | 81 |
| Participant-32 | 83 |
| Participant-32 | 80 |
| Participant-32 | 80 |
| Participant-30 | 88 |
| Participant-26 | 114 |
| NEW SUBJECT- 30 | 90 |

As seen from FIG. 20, there is a consistent, inverse relationship between the red light fraction and the VLS skin tone value. By extrapolating the observations, the value of VLS skin tone (T) for a device measured red-light fraction (F) value can be determined by the equation:

Y=Reflection of red light [F]

X=Skin color [T]

Y=265−5.8X

F=265−5.8*T

F=265−6T

To confirm, a random subject with VLS skin tone 30 [NEW SUBJECT-30] participated in the observational experiment. The processor 4 determined a red fraction (F) value for this subject at 90. Using the equation, the device estimated VLS skin tone for the subject a 30.17 [T=30.17], very close to the clinically estimated VLS skin tone value of 30.

The described embodiments and examples of the present disclosure are intended to be illustrative rather than restrictive and are not intended to represent every embodiment or example of the present disclosure. While the fundamental novel features of the disclosure as applied to various specific embodiments thereof have been shown, described, and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

1. A device comprising:
a plurality of light emitting diodes configured to emit light at a wavelength in a range of (1) about 430 nm to about 470 nm, (2) about 505 nm to about 545 nm, and (3) about 525 nm to about 565 nm towards a surface of a mammal's skin surface;
one or more sensors configured to detect an amount of reflected light in a range of (1) about 430 nm to about 470 nm, (2) about 505 nm to about 545 nm, and (3) about 525 nm to about 565 nm, wherein the detected, reflected light is the emitted light that passes through at least a portion of the mammal's skin and is at least partially reflected towards the one or more sensors, wherein the one or more sensors are configured to output a signal comprising the amount of the detected, reflected light in the ranges of (1) about 430 nm to about 470 nm, (2) about 505 nm to about 545 nm, and (3) about 525 nm to about 565 nm; and
a processor configured to:
send a signal of emitted light at the wavelength in the range of (1) about 430 nm to about 470 nm, a signal of emitted light at the wavelength in the range of (2) about 505 nm to about 545 nm, and a signal of emitted light at the wavelength in the range of (3) about 525 nm to about 565 nm,
receive the signal of the amount of the detected, reflected light in the range of (1) about 430 nm to about 470 nm, a signal of the amount of the detected, reflected light in the range of (2) about 505 nm to about 545 nm, and a signal of the amount of the detected, reflected light in the range of (3) about 525 nm to about 565 nm,
determine a Z ratio of reflected light, wherein the Z ratio is a percentage of detected, reflected light in the range of about 430 nm to about 470 nm as compared to the light emitted in the range of about 430 nm to about 470 nm,
determine an E1 ratio, wherein the E1 ratio is a percentage of detected, reflected light in the range of about 505 nm to about 545 nm as compared to the light emitted in the range of about 505 nm to about 545 nm,
determine an E2 ratio, wherein the E2 ratio is a percentage of detected,
reflected light in the range of about 525 nm to about 565 nm as compared to the light emitted in the range of about 525 nm to about 565 nm,
determine an E value by adding the E1 ratio to the E2 ratio,
determine a bilirubin value by dividing the E value by the Z ratio; and
output the bilirubin value.

2. The device of claim 1, wherein
the Z ratio is the percentage of detected, reflected light in the range of about 440 nm to about 460 nm as compared to the light emitted in the range of about 440 nm to about 460 nm,
the E1 ratio is the percentage of detected, reflected light in the range of about 515 nm to about 535 nm as compared to the light emitted in the range of about 515 nm to about 535 nm, and
the E2 ratio is the percentage of detected, reflected light in the range of about 535 nm to about 555 nm as compared to the light emitted in the range of about 535 nm to about 555 nm.

3. The device of claim 1, wherein
the Z ratio is the percentage of detected, reflected light in the range of about 450 nm as compared to the light emitted in the range of about 450 nm,
the E1 ratio is the percentage of detected, reflected light in the range of about 525 nm as compared to the light emitted in the range of about 525 nm, and
the E2 ratio is the percentage of detected, reflected light in the range of about 545 nm as compared to the light emitted in the range of about 545 nm.

4. The device of claim 1, wherein the bilirubin value is output to a display that is configured to display text and/or images of the value.

5. The device of claim 4, wherein the display is configured to receive a touch input.

6. The device of claim 1, further comprising an optical shield.

7. The device of claim 6, wherein the optical shield comprises at least two alternating layers.

8. The device of claim 7, wherein a first layer of the at least two alternating layers comprises a foil material, and wherein a second layer of the at least two alternating layers comprises a sheet material.

9. A device comprising:
a plurality of light emitting diodes configured to emit light at a wavelength in a range of about 540 nm to about 700 nm towards a surface of a mammal's skin surface;
one or more sensors configured to detect an amount of reflected light in a range of about 600 nm to about 700 nm, wherein the detected, reflected light is the emitted light that passes through at least a portion of the mammal's skin and is at least partially reflected towards the one or more sensors, wherein the one or more sensors are configured to output a signal comprising the amount of the detected, reflected light in the range of about 600 nm to about 700 nm; and
a processor configured to:
send a signal of emitted light at the wavelength in the range of about 540 nm to about 700 nm,
receive the signal of the amount of the detected, reflected light in the range of about 600 nm to about 700 nm,
determine a red pixel value of the detected, reflected light,
determine a melanin value based on the determined red pixel value; and
output values of the total melanin value.

10. The device of claim 6, wherein the melanin value is output to a display that is configured to display text and/or images of the value.

11. The device of claim 7, wherein the display is configured to receive a touch input.

12. The device of claim 9, further comprising an optical shield.

13. The device of claim 12, wherein the optical shield comprises at least two alternating layers.

14. The device of claim 13, wherein a first layer of the at least two alternating layers comprises a foil material, and wherein a second layer of the at least two alternating layers comprises a sheet material.
